(19) United States
(12) Patent Application Publication
(10) Pub. No.: US 2021/0153774 A1
(43) Pub. Date: May 27, 2021

(54) Single Patient Use Throwaway Device for Recording Respiratory Flow Rates

(71) Applicant: Shabbir Husain Moiyed-Tailor, Bangalore (IN)

(72) Inventor: Shabbir Husain Moiyed-Tailor, Bangalore (IN)

(73) Assignee: Aerobit Labs Pvt. Ltd., Pune (IN)

(21) Appl. No.: 16/695,342

(22) Filed: Nov. 26, 2019

(51) Int. Cl. A61B 5/087; B29C 45/00

(52) U.S. Cl. CPC A61B 5/0871; B29L 2031/753; B29C 45/0001

(57) ABSTRACT

The present disclosure relates to a throwaway device to check and measure respiratory flow rates. The device 100 incorporates a first deflector 104, a blade 108 and a second deflector 106 enclosed inside a housing 102. The first deflector 104 configured at a front end of the housing 102 to facilitate inflow of air inside the housing 102. The second deflector 106 is configured at the rear end of the housing 102 to facilitate outflow of air. The blade 108 is having two conical ends 110 at two opposite edges. The blade 108 positioned between the first deflector 104 and the second deflector 106 which is configured to rotate about a rotational axis A-A′. A mouth piece 112 is removable attached at the front end of the housing 102. The blade, housing and the two deflectors are made of different plastic materials using multiple sessions of injection molding process.

### TECHNICAL FIELD

The present disclosure relates to a single patient use throwaway device, more particularly, the present disclosure relates to construction and features of a throwaway turbine for Spirometer, Peak Flow meters and Pulmonary Function Test (PFT) instruments to check and measure respiratory flow rates.

### BACKGROUND

Background description includes information that may be useful in understanding the present invention. It is not an admission that any of the information provided herein is prior art or relevant to the presently claimed invention, or that any publication specifically or implicitly referenced is prior art.

In present world, life of people is changing rapidly with rapid increase in population. This increase in population has led the pollution level to reach a level which is very dangerous and beyond safe limits for human being and animals. Pollution is directly affecting people of every age, and air pollution is one of the major factors that are causing various health related issues in people. Air pollution is directly affecting the respiratory system of normal people as well as the people already having respiratory disorders.

Early detection of respiratory disorders is very important in prevention and treatment of such respiratory disorders. Otherwise, these respiratory disorders may become severe and incurable after a certain period of time.

Various techniques and instruments are present in the market for detection of respiratory disorders. One of the most common techniques has been measurement of the volume of air that can be exhaled or inhaled from completely inflated lungs of a person. And this measurement is a vital sign for studying lung function and other respiratory disorders associated with the person. Spirometer, Peak Flow meters and Pulmonary Function Test (PFT) are instruments which are widely used by doctors and patients in clinics, hospitals, field survey, clinical trials and home for studying lung function and for examining respiratory disorders in people (patients). These instruments measure the volume of air that can be exhaled or inhaled from completely inflated or deflated lungs of a person. This measurement is useful in studying respiratory problems such as asthma, COPD, etc, and to evaluate possible occupational pulmonary disorders.

Various such instruments are available in the market, but these instruments are costly to be afforded by every people and/or bulky to be carried at different places.

These instruments include a device (also sometimes referred to as turbine) as a primary tester for measuring the respiratory flow rates (i.e., the inspired or expired air volume or speed or flux of the air) of a user. These devices facilitates inflow and outflow of air exhaled/inhaled by the user, inside the instruments for example, Spirometer, Peak Flow meters and Pulmonary Function Test (PFT), to transform and process the measured respiratory flow rates of the user for further analysis of the user's breathing functionality.

In addition, respiratory diseases are easily spreadable and such devices are required to be replaced after either single usage or sterilized in order to prevent other users from infection and prevent spreading of any diseases. Replacement of such devices at present is unaffordable. Sterilization is also an extremely time consuming and costly effort that needs to be undertaken by the clinician.

There is, therefore, a need to provide a portable, $$fordable and throwaway device for such instruments to study lung function and detect respiratory disorders that overcomes above stated drawbacks of conventional instruments as well as the devices of the instruments. It would be advantageous to provide a single patient use throwaway device at an extremely affordable price versus a bulky, costly reusable device which requires sterilization after every use.

### OBJECTS OF THE PRESENT DISCLOSURE

Some of the objects of the present disclosure, which at least one embodiment herein satisfies are as listed herein below.

It is an object of the present disclosure to provide a throwaway device for spirometer instruments to measure respiratory flow rate of user.

It is an object of the present disclosure to provide a portable throwaway device for Spirometer, Peak Flow meters and Pulmonary Function Test (PFT) instruments to measure respiratory flow rate of user.

It is an object of the present disclosure to provide a portable throwaway device for Spirometer, Peak Flow meters and Pulmonary Function Test (PFT) to measure respiratory flow rate of user for examining lung function and detection of respiratory disorders.

### SUMMARY

The present disclosure relates to a single patient use throwaway device, more particularly, the present disclosure relates to construction and features of a throwaway turbine for Spirometer, Peak Flow meters and Pulmonary function test (PFT) instrument to check and measure respiratory flow rates.

An aspect of the present disclosure pertains to a throwaway device for checking respiratory flow rates, constructed using multiple printing sessions of injection molding process, the throwaway device may include a housing; a first deflector, which may be configured at a first end of the housing, the first deflector may be configured to facilitate air flow into or from the housing; a second deflector, which may be configured at a second end of the housing, the second deflector may be configured to facilitate an outflow/inflow of air from/to the housing; and a blade, which may be configured inside the housing between the first deflector and the second deflector, the blade may be provided with two conical ends on two opposite edges of the blade about a rotational axis that extends through a center of the first deflector and the second deflector, wherein the blade may be configured to rotate about the rotational axis.

In an aspect, the blade may be fabricated using a plastic material different from the housing, the first deflector, and the second deflector, and wherein the plastic material having mold flow rates between 65 g/10 min to 75 g/10 min (as per ISO 1133) and a density between 895 Kg/m³ to 910 Kg/m³ (as per ISO 1183). More preferably and specifically, the mold flow rate is 70 g/10 min and the density is 905 Kg/m³.

In an aspect, the blade is fabricated using a medical grade biocompatible thermo plastic material different from the housing, the first deflector, and the second deflector.

In an aspect, the housing, the first deflector and the second deflector is made of a medical grade thermo plastic material different to blade material.

In an aspect, the housing, the first deflector and the second deflector and the blade is made up of biocompatible thermo plastic material.

In an aspect, the blade comprises at two edges, each of the edges having a thickness of 100 micron to 140 micron. More preferably and specifically, the thickness is 110 micron.

In an aspect, each of the two conical ends having a diameter of 500 micron to 600 micron. More preferably and specifically, the diameter is 530 micron.

In another aspect, each of the two conical ends of the blade may be beveled at an angle between 24 and 26° with the rotational axis.

In yet another aspect, the throwaway device may comprise a cardboard mouth piece removably coupled at the first end of the housing.

In an aspect, the throwaway device may be adapted to be configured with a Spirometer, Peak Flow meters and Pulmonary Function Test (PFT) instruments.

Compared with a currently existing disposable turbines and/or reusable turbines which can be produced and realized in one single printing using injection production phase out of plastic material preferably making use of always the same raw material, in the embodiments of the present invention, each of the components i.e., the housing, the first deflector, the second deflector, and the blade are constructed using multiple printing sessions of injection molding process, using different materials making them stronger and sustainable during operating conditions to provide accurate and effective results.

Also, since the multiple printing sessions are required during injection molding process, it may be appreciated that, in case of defect in any one component i.e., the housing or the first deflector or the second deflector or the blade, the whole process need not be stopped, but only the step that failed has to be stopped and replacements needs to be made.

Further, as compared with currently existing disposable turbines, the blade according to the embodiments of the present invention comprises at two edges and two conical ends wherein each of the edges having a thickness of 110 micron and each of the two conical ends having a diameter of 530 micron. With this structure the present invention enables to perfectly balance the weight of the blade by reducing it from the competitive blade. Lower density and lower weight structure gives the blade a more efficient movement.

Further, as compared with currently existing devices (for example, turbines of the Spirometer, Peak Flow meters and Pulmonary Function Test (PFT)), the first deflector and/or the second deflector according to the embodiments of the present invention include a helical structure to provide swirl effect to the air entering the housing of the device. This swirled inflow/outflow of air can facilitate the rotation of the blade above 1100 rotations per sec.

Various objects, features, aspects and advantages of the inventive subject matter will become more apparent from the following detailed description of preferred embodiments, along with the accompanying drawing figures in which like numerals represent like components.

Within the scope of this application it is expressly envisaged that the various aspects, embodiments, examples and alternatives set out in the preceding paragraphs, in the claims and/or in the following description and drawings, and in particular the individual features thereof, may be taken independently or in any combination. Features described in connection with one embodiment are applicable to all embodiments, unless such features are incompatible.

### BRIEF DESCRIPTION OF DRAWINGS

The accompanying drawings are included to provide a further understanding of the present disclosure, and are incorporated in and constitute a part of this specification. The drawings illustrate exemplary embodiments of the present disclosure and, together with the description, serve to explain the principles of the present disclosure. The diagrams are for illustration only, which thus is not a limitation of the present disclosure.

In the figures, similar components and/or features may have the same reference label. Further, various components of the same type may be distinguished by following the reference label with a second label that distinguishes among the similar components. If only the first reference label is used in the specification, the description is applicable to any one of the similar components having the same first reference label irrespective of the second reference label.

FIG. 1 illustrates an exploded view of the proposed throwaway device, in accordance with an embodiment of the present disclosure.

FIG. 2 illustrates the proposed assembled throwaway device, in accordance with an embodiment of the present disclosure.

### DETAILED DESCRIPTION

The following is a detailed description of embodiments of the disclosure depicted in the accompanying drawings. The embodiments are in such detail as to clearly communicate the disclosure. However, the amount of detail offered is not intended to limit the anticipated variations of embodiments; on the contrary, the intention is to cover all modifications, equivalents, and alternatives falling within the spirit and scope of the present disclosure as defined by the appended claims.

In the following description, numerous specific details are set forth in order to provide a thorough understanding of embodiments of the present invention. It will be apparent to one skilled in the art that embodiments of the present invention may be practiced without some of these specific details.

If the specification states a component or feature “may”, “can”, “could”, or “might” be included or have a characteristic, that particular component or feature is not required to be included or have the characteristic.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

The use of “including”, “comprising” or “having” and variations thereof herein is meant to encompass the items listed thereafter and equivalents thereof as well as additional items. The terms “a” and “an” herein do not denote a limitation of quantity, but rather denote the presence of at least one of the referenced item. Further, the use of terms “first”, “second”, and “third”, and the like, herein do not denote any order, quantity, or importance, but rather are used to distinguish one element from another.

The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all groups used in the appended claims.

Exemplary embodiments will now be described more fully hereinafter with reference to the accompanying drawings, in which exemplary embodiments are shown. This invention may, however, be embodied in many different forms and should not be construed as limited to the embodiments set forth herein. These embodiments are provided so that this disclosure will be thorough and complete and will fully convey the scope of the invention to those of ordinary skill in the art. Moreover, all statements herein reciting embodiments of the invention, as well as specific examples thereof, are intended to encompass both structural and functional equivalents thereof. Additionally, it is intended that such equivalents include both currently known equivalents as well as equivalents developed in the future (i.e., any elements developed that perform the same function, regardless of structure).

The present disclosure relates to a single patient use throwaway device, more particularly, the present disclosure relates to construction and features of a throwaway turbine for Spirometer, Peak Flow meters and Pulmonary function test (PFT) instrument to check and measure respiratory flow rates.

An aspect of the present disclosure pertains to a throwaway device for checking respiratory flow rates, constructed using multiple printing sessions of injection molding process, the throwaway device may include a housing; a first deflector, which may be configured at a first end of the housing, the first deflector may be configured to facilitate air flow into or from the housing; a second deflector, which may be configured at a second end of the housing, the second deflector may be configured to facilitate an outflow/inflow of air from/to the housing; and a blade, which may be configured inside the housing between the first deflector and the second deflector, the blade may be provided with two conical ends on two opposite edges of the blade about a rotational axis that extends through a center of the first deflector and the second deflector, wherein the blade may be configured to rotate about the rotational axis.

In an aspect, the blade may be fabricated using a plastic material different from the housing, the first deflector, and the second deflector, and wherein the plastic material having mold flow rates between 65 g/10 min to 75 g/10 min (as per ISO 1133) and a density between 895 Kg/m³ to 910 Kg/m³ (as per ISO 1183). More preferably and specifically, the mold flow rate is 70 g/10 min and the density is 905 Kg/m³.

In an aspect, the blade is fabricated using a medical grade biocompatible thermo plastic material different from the housing, the first deflector, and the second deflector.

In an aspect, the housing, the first deflector and the second deflector is made of a medical grade thermo plastic material different to blade material.

In an aspect, the housing, the first deflector and the second deflector and the blade is made up of biocompatible thermo plastic material.

In an aspect, the blade comprises at two edges, each of the edges having a thickness of 100 micron to 140 micron. More preferably and specifically, the thickness is 110 micron.

In an aspect, each of the two conical ends having a diameter of 500 micron to 600 micron. More preferably and specifically, the diameter is 530 micron.

In another aspect, each of the two conical ends of the blade may be beveled at an angle between 24 and 26° with the rotational axis.

In yet another aspect, the throwaway device may comprise a cardboard mouth piece removably coupled at the first end of the housing.

In an aspect, the throwaway device may be adapted to be configured with a Spirometer, Peak Flow meters and Pulmonary Function Test (PFT) instruments.

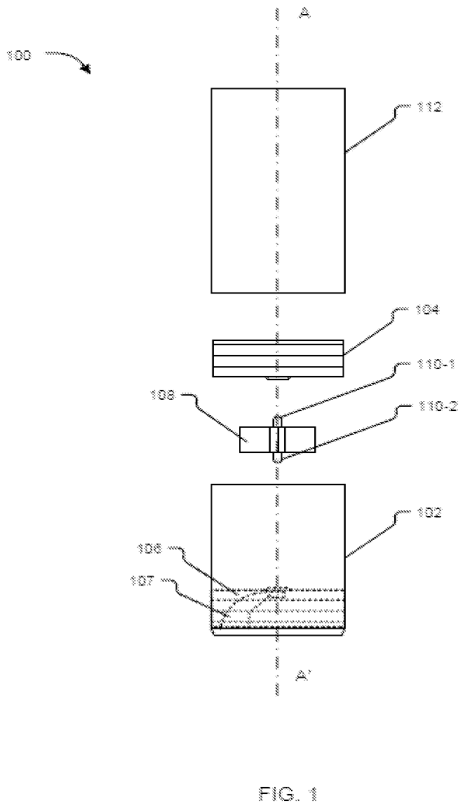

FIG. 1 illustrates an exploded view of the proposed throwaway device, in accordance with an embodiment of the present disclosure.

As illustrated, in an embodiment, the proposed throwaway device 100 (also designated as “throwaway device” or “turbine”, herein) for checking the respiratory flow rate can include a housing 102, a first deflector 104, a second deflector 106, a blade 108 with two conical ends, and a mouth piece 112. The throwaway device 100 includes the housing 102, which can enclose the first deflector 104 at a front end of the housing 102 and the second deflector 106 at a second end of the housing 102. The first defector 104 can facilitate an inflow of air inside the housing 102. The second deflector 106 can facilitate an outflow of air from the housing 102.

In an exemplary embodiment, the housing 102 can be a cylindrical pipe shaped structure. The first deflector 104 and the second deflector 106 can be circular or cylindrical shaped, positioned at two opposite ends of the cylindrical pipe shaped housing 102.

In an embodiment, the housing 102 can include the blade 108 configured inside the housing 102 between the first deflector 104 and the second deflector 106. The blade 108 can be provided with two conical ends 110-1 and 110-2 (collectively referred to as conical ends 110, herein) on two opposite edges of the blade 108 extending about a rotational axis A-A′ that extends through a center of the first deflector 104 and the second deflector 106. The blade 108 can be configured to rotate about the rotational axis A-A′.

In an embodiment, the blade 108, the housing 102 and the deflectors (104, 106) of the proposed device 100 can be made of different suitable materials to achieve cost effective and optimal result compared to other conventional other devices/turbines having each components made of same type of material. Further, in another embodiment, the proposed throwaway device 100 can be made using multiple sessions of injection molding process for improved strength and to facilitate easier replacement of each components of the proposed device 100 without replacing the complete device.

In an embodiment, the throwaway device 100 can include a mouth piece 112 removably coupled at the first end of the housing 102. The mouth piece 112 can facilitate a user to blow/exhale air inside the proposed device 100.

In an embodiment, the housing 102 comprises a slot 107 to enable the housing 102 to engage with Spirometer, Peak Flow meters and Pulmonary Function Test (PFT) instruments to check and measure respiratory flow rates.

In an embodiment, the blade 108 can be fabricated using a material having mold flow rates between 65 g/10 min to 75 g/10 min and a density between 895 Kg/m³ to 910 Kg/m³, but not limited to the likes. More preferably and specifically, the mold flow rate is 70 g/10 min and the density is 905 Kg/m³.

In an embodiment, each of the two conical ends 110 of the blade 108 can be bevelled at an angle of 16° with respect to the rotational axis of the blade 108.

In an embodiment, the housing, the first deflector and the second deflector is made of a medical grade polycarbonate.

In an embodiment, the blade is fabricated using a medical grade biocompatible polypropylene different from a material used for fabricating the housing, the first deflector, and the second deflector.

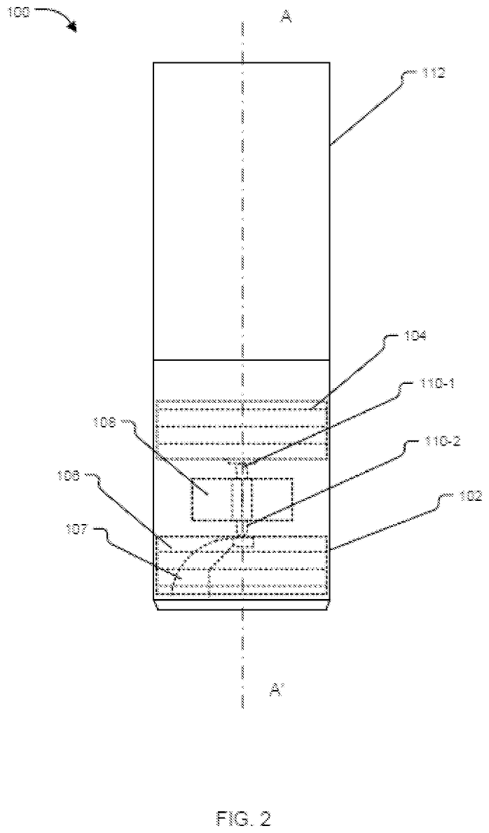

FIG. 2 illustrates the proposed assembled throwaway device, in accordance with an embodiment of the present disclosure.

As illustrated, in an embodiment, the proposed device in an assembled state can include the blade 108 being rotatably configured between the first deflector 104 and the second deflector 106 inside the housing 102 such that the blade 108 can rotate about a rotational axis that extends through a center of the first deflector 104 and the second deflector 106. In an embodiment, the first deflector 104 can include a first cavity at the centre of the first deflector 104, and the second deflector 106 can include a second cavity at the centre of the second deflector 106. Each of the first cavity and the second cavity can be configured to accommodate one of the two conical ends of the blade 108. For instance, the conical end 110-1 can be rotatably configured at the center of the first cavity of the first deflector 104, and the conical end 110-2 can be rotatably configured at the center of the second cavity of the second deflector 106.

In an embodiment, the mouth piece 112 can be removably coupled at the first end of the housing 102 preferably in vicinity with the first deflector 104. In an embodiment, the spirometer device 100 can be adapted to be configured with various Spirometer, Peak Flow meters and Pulmonary Function Test (PFT) instruments.

In an exemplary embodiment, the first deflector 104 can include a plurality of first vanes extending outward from the first cavity towards a circumference of the first deflector 104, and the second deflector 106 can include a plurality of second vanes extending outward from the second cavity towards a circumference of the second deflector 106. In another exemplary embodiment, the plurality of first vanes and the plurality of second vanes of the first deflector 104 and the second deflector 106 can have curved profile.

In an embodiment, the first deflector 104 can be configured to restrict flow of foreign bodies inside the housing 102 of the device.

In an embodiment, the first deflector 104 can include a helical structure to provide swirl effect to the air entering the housing 102 of the device 100. This swirled inflow of air can facilitate the rotation of the blade 108 above 1000 rotations per sec. The second deflector 106 can then facilitate the air to flow out of the housing 102.

In an implementation, a user can exhale/blow air into the proposed device 100 using through the mouth piece 112. The exhaled air can then pass through the first deflector 104, which can facilitate flow of exhaled air inside the housing 102. The first deflector 104 can provide swirl effect to the inflow of air, which then passes around the blade 108 to facilitate rotation of the blade 108 about the rotational axis. Finally, the air can pass through the second deflector 106, which can facilitate flow of air outside the housing 102. The number of rotations of the blade 108 can correspond to the respiratory flow rate (i.e., the inspired or expired air volume or speed or flux of the air) of the user. The respiratory flow rate of the user can be associated with the user's breathing functionality and lung function. The measured flow rate facilitates detection of respiratory disorders using the instruments configured with the throwaway device 100.

As used herein, and unless the context dictates otherwise, the term “coupled to” is intended to include both direct coupling (in which two elements that are coupled to each other or in contact each other) and indirect coupling (in which at least one additional element is located between the two elements). Therefore, the terms “coupled to” and “coupled with” are used synonymously. Within the context of this document terms “coupled to” and “coupled with” are also used euphemistically to mean “communicatively coupled with” over a network, where two or more devices are able to exchange data with each other over the network, possibly via one or more intermediary device.

Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

While the foregoing describes various embodiments of the invention, other and further embodiments of the invention may be devised without departing from the basic scope thereof. The scope of the invention is determined by the claims that follow. The invention is not limited to the described embodiments, versions or examples, which are included to enable a person having ordinary skill in the art to make and use the invention when combined with information and knowledge available to the person having ordinary skill in the art.

### Advantages of the Invention

The present invention provides a device for Spirometer, Peak Flow meters and Pulmonary Function Test (PFT) instruments to measure respiratory flow rate of user.

The present invention provides a portable device for Spirometer, Peak Flow meters and Pulmonary Function Test (PFT) instruments to measure respiratory flow rate of user.

The present invention provides a portable turbine device for Spirometer, Peak Flow meters and Pulmonary Function Test (PFT) instrument to measure respiratory flow rate of user for examining lung function and detection of respiratory disorders.

The present invention provides a throwaway and portable turbine device for Spirometer, Peak Flow meters and Pulmonary Function Test (PFT) instruments to measure respiratory flow rate examining lung function and detection of respiratory disorders.

The present invention, due to blade's low density and lighter weight, provides more rotations as compared to the blades used in conventional Spirometer, Peak Flow meters and Pulmonary Function Test (PFT) instruments and hence it provides more accurate results

1-4. (canceled)

5. The throwaway device as claimed in claim 11, wherein the housing, the first deflector, the second deflector and the blade are made up of a biocompatible material.

6. The throwaway device as claimed in claim 11, wherein the blade comprises at least two edges, each of the edges having a thickness in the range between 100-140 μm.

7. The throwaway device as claimed in claim 11, wherein each of the two conical ends has a diameter in the range between 500-600 μm.

8. The throwaway device as claimed in claim 11, wherein the first deflector and the second deflector comprise a helical structure to provide a swirl effect to the air entering the housing of the device to facilitate the rotation of the blade.

9. (canceled)

10. (canceled)

11. A throwaway device for checking respiratory flow rates, constructed using multiple printing sessions of an injection molding process, the throwaway device comprising:
a housing;
a first deflector configured at a first end of the housing, the first deflector configured to facilitate inflow or outflow of air into or from the housing;
a second deflector configured at a second end of the housing to facilitate outflow or inflow of air from or into the housing;
a blade configured inside the housing between the first deflector and the second deflector, wherein the blade has two conical ends, each on opposite sides of the blade, to provide a rotational axis that extends through a center of the first deflector and the second deflector;
wherein the blade comprises a plastic material having a mold flow rate of 65-75 g/10 min and density of 895-910 Kg/m3;
wherein the plastic material for the blade is different from the material of which the housing, the first deflector, and the second deflector comprises;
wherein the plastic material for the blade is of lower density than that of the housing, the first deflector, and the second deflector.

12. The throwaway device as claimed in claim 11, wherein the plastic material for the blade is polypropylene.

13. The throwaway device as claimed in claim 12, wherein the blade comprises at least two edges, each of the edges having a thickness in the range between 100-140 μm.

14. The throwaway device as claimed in claim 13, wherein each of the two conical ends has a diameter in the range between 500-600 μm.

15. The throwaway device as claimed in claim 14, wherein the first deflector and the second deflector comprise a helical structure to provide a swirl effect to the air entering the housing of the device to facilitate the rotation of the blade.
